(19) United States
(12) Patent Application Publication
(10) Pub. No.: US 2025/0057610 A1
(43) Pub. Date: Feb. 20, 2025

(54) ROBOTIC SYSTEMS AND INSTRUMENTS

(71) Applicant: EndoQuest Robotics, Inc., Houston, TX (US)

(72) Inventors: Yongman Park, Houston, TX (US); Daniel Kim, Houston, TX (US); Raymond Lee, Houston, TX (US); Sungwoo Cho, Houston, TX (US); Dongsuk Shin, Houston, TX (US)

(73) Assignee: EndoQuest Robotics, Inc., Houston, TX (US)

(21) Appl. No.: 18/933,901

(22) Filed: Oct. 31, 2024

Related U.S. Application Data
(63) Continuation-in-part of application No. 18/134,689, filed on Apr. 14, 2023, now Pat. No. 12,144,571.
Application No. 18/134,689 is a continuation of application No. PCT/US2022/051265, filed on Nov. 29, 2022.
(60) Provisional application No. 63/284,298, filed on Nov. 30, 2021.

(51) Int. Cl. A61B 34/30; A61B 17/00

(52) U.S. Cl. CPC A61B 34/30; A61B 2017/00318

(57) ABSTRACT

A force transmission system for a robotically controlled medical device includes a pushing actuator adapted and configured to be pushed distally by a robotic instrument controller, a pivot, and a reverse linkage rotatable about the pivot. The reverse linkage includes a first portion extending between the pivot and the pushing actuator, and operably engaged with the pushing actuator, a second portion extending from the pivot away from the first portion, adapted to engage a control wire of the robotically controlled medical device and pull the control wire proximally in response to distal movement of the pushing actuator, and a distal surface of the second portion having an arcuate surface adapted maintain an axial position of the control wire throughout a range of motion thereof.

### CROSS-REFERENCE TO RELATED APPLICATIONS

This application is a continuation-in-part of U.S. patent application Ser. No. 18/134,689 filed Apr. 14, 2023, which is a continuation of International Patent Application No. PCT/US2022/051265 filed Nov. 29, 2022, which claims priority to and the benefit of U.S. Provisional Application No. 63/284,298, filed Nov. 30, 2021. The entire contents of each of the foregoing are incorporated by reference herein, in their entirety.

### TECHNICAL FIELD

This disclosure is directed to various aspects of surgical robots utilizing a flexible access port or steerable overtube, which are particularly suited for use in endoluminal (endolumenal) surgical procedures. More particularly, this disclosure relates to robotically assisted transoral, transesophageal, transumbilical, intragastric, transanal and transvaginal endoscopic surgical procedures, techniques, and treatments, sometimes referred to as Natural Orifice Translumenal Endoscopic Surgery (NOTES). This disclosure also relates to Single Incision Laparoscopic Surgery (SILS), Single Port Access (SPA) surgery, Natural Orifice Trans-Umbilical Surgery (NOTUS), Laparo-Endoscopic Single-site Surgery (LESS), One Port Umbilical Surgery (OPUS), Single Port Incisionless Conventional Equipment-utilizing Surgery (SPICES), and Single Access Site Surgical Endoscope (SASSE) procedures.

Additionally, this disclosure is directed to various aspects of robots designed for performing functions in confined spaces, including industrial applications. Specifically, the systems, devices and related methods of the present invention can advantageously be applied to various nonmedical fields, such as industrial robots and remotely operated vehicles, including those used in outer space or deep-sea environments, for example in oil and gas exploration. The invention is particularly advantageous to fields requiring precise control in performing complex tasks in confined and/or difficult-to-reach structures (such as within long conduits), or in situations where access requires navigation around or through existing structures, including curved structures.

### BACKGROUND OF THE INVENTION

Minimally invasive surgical procedures such as endoluminal surgery and single-site laparoscopic surgery are known in the art and provide many benefits over traditional open or multi-port laparoscopic surgical procedures. Endoluminal surgical procedures are performed endoscopically within hollow organs using typical surgical techniques, such as dissection, suturing, cutting, and stapling. These procedures may be performed trans-orally within the upper gastrointestinal (GI) tract, trans-anally within the lower GI tract, or trans-vaginally within the abdominal cavity. A significant benefit of endoluminal surgery is that no skin incision is needed to access to the surgical site within a patient's natural lumen. This can dramatically reduce patient recovery time and can improve procedural safety. Similarly, single-site or single incision laparoscopic surgical procedures are typically performed within a patient's abdominal cavity or thoracic cavity through a single incision. This can also reduce patient recovery time and trauma, as multiple incisions are avoided, providing greater flexibility in incision location.

Robotic systems are also known in the art and have been used to perform industrial tasks. Moreover, robotic surgical systems are also known in the art and have been used to perform medical and surgical procedures, such as endoluminal and single site surgical procedures. An example of such a system is disclosed, for example, in commonly assigned U.S. Patent Application Publication 2023/0285098. This flexible robotic system includes a patient cart or console with a multi-axis positioning system, and it employs a steerable overtube assembly having a plurality of working channels for introducing surgical devices to a surgical site. The overtube assembly is disclosed in commonly assigned U.S. Patent Application Publication 2023/0210618, which is also incorporated herein by reference in its entirety. Exemplary surgical devices and end effectors or tools that can be introduced to a surgical site through a working channel of the steerable overtube assembly are disclosed in commonly assigned U.S. Patent Application Publication 2023/0248419, the disclosure of which is incorporated herein by reference in its entirety.

Systems, devices and methods in accordance with the invention can incorporate or utilize aspects of devices, systems and methods disclosed in the following, each of which is incorporated herein by reference in its entirety: Master Control Systems for Robotic Surgical Systems, as described in U.S. Patent Application Publication 2023/0285090, and/or User Interfaces For Surgical Robotic Systems, as described in U.S. patent application Ser. No. 18/920,441, and/or Position Control for Patient Console, as described in U.S. Patent Application Publication 2023/0363842, and/or Safety Hand Sensor for Robotic Surgical System, as described in U.S. Patent Application Publication 2023/0210621, and/or Display Systems for Robotic Surgical Systems, as described in U.S. Patent Application Publication 2023/0248450, and/or Wire Elongation Compensation System, as described in U.S. Patent Application Publication 2023/0285099, and/or Controller Arrangements for Robotic Surgical Systems, as described in U.S. Patent Application Publication 2023/0248457, and/or Barrier Drape Adapters for Robotic Surgical Systems, as described in U.S. Patent Application Publication 2023/0363847, and/or Force Transmission Systems for Robotically Controlled Medical Devices, as described in U.S. Patent Application Publication 2023/0255702, to which this application claims priority, and/or Systems And Method for Trans-Luminal Introduction Of A Medical Device, as described in U.S. Patent Application Publication 2023/0355221, and/or Robotic Medical System Drape Adapter Assemblies, as described in U.S. patent application Ser. No. 18/415,502, and/or Valve Assembly for Sealing an Instrument Channel on a Robotic Surgical System, as described in U.S. patent application Ser. No. 18/535,425, and/or Support Assembly for Holding a Videoscope on a Robotic Surgical System, as described in U.S. patent application Ser. No. 18/596,171, and/or Robotically Assisted Endoluminal Surgical Procedures, as described in U.S. Patent Application 63/641,114, and/or Robotically Assisted Single-Incision Endoscopic Surgical Procedures, as described in U.S. Patent Application 63/641,165, and/or Posable Patient Cart for Performing Robotically Assisted Surgical Procedures, as described in U.S. Patent Application No. 63/677,648, and/or Roll and Pitch Module for Robotic Surgical System, as described in U.S. Patent Application No. 63/677,576, and/or Central Drive Unit (CDU) and Translation Module for Robotic Surgical System, as described in U.S. Patent Application No. 63/677,614, and/or Cart and Tower Module for Robotic Surgical System, as described in U.S. Patent Application No. 63/677,648, and/or Mechanical Apparatus for volume effective bipolar energy instrument end effector open/close mechanism, as described in U.S. patent application Ser. No. 18/790,627, and/or Surgical Apparatus, as described in U.S. Pat. No. 11,607,238, and/or Surgical Apparatus, as described in U.S. Patent Publication Number 2021/0275266, and/or End Effector and End Effector Drive Apparatus, as described in U.S. Patent Publication Number 2020/0397456, and/or Seven Degree of Freedom Positioning Device for Robotic Surgery, as described in U.S. Patent Publication Number 2024/0058079.

Applicant recognizes a need in the art for improved robotic surgical systems, devices, methods, controls, and components, especially those configured for endoluminal and single-site surgery. The present disclosure provides improvements in robotic surgical systems, devices, instruments, methods, controls, components, and other accessories and ancillary components, among others, as will be appreciated.

### SUMMARY OF THE INVENTION

The purpose and advantages of the illustrated embodiments will be set forth in and apparent from the following description. Additional benefits thereof will be realized and attained through the devices, systems, methods, controls, components, instruments and other accessories and ancillary components highlighted in the written description, claims, and the appended drawings.

To achieve these and other advantages, and in accordance with the purpose of the illustrated embodiments, one aspect of the present invention relates to a force transmission system for a robotically controlled medical device including a pushing actuator adapted and configured to be pushed distally by a robotic instrument controller, a pivot, and a reverse linkage rotatable about the pivot. The reverse linkage includes a first portion extending between the pivot and the pushing actuator, and operably engaged with the pushing actuator, a second portion extending from the pivot away from the first portion, adapted to engage a control wire of the robotically controlled medical device and pull the control wire proximally in response to distal movement of the pushing actuator, and a distal surface of the second portion having an arcuate surface adapted to maintain an axial position of the control wire throughout a range of motion of the pushing actuator.

The arcuate surface can be adapted to maintain a radial position of the control wire with respect to a central axis of the robotically controlled medical device. The arcuate surface can be defined by a constant radius from the pivot. The arcuate surface can be provided with a groove to guide the control wire and maintain operable engagement therewith.

The force transmission system can further include the control wire, the control wire having first and second ends, a first end thereof being secured to the second portion of the reverse linkage. The control wire can be engaged with an aperture formed in the reverse linkage. The pushing actuator can be a linear pushing actuator and the first portion of the reverse linkage can be provided with a sliding joint between the pivot and a point of engagement with the pushing actuator, adapted to adjust to a changing radius between the pushing actuator and the pivot through a range of motion thereof. The force transmission system can further include a base, the pushing actuator extending through the base and the pivot secured by the base. The force transmission system can further include at least two reverse motion devices associated with each of at least two respective control wires, adapted and configured to antagonistically operate a first motion and a second opposing motion of a function of the robotically controlled medical device. The function can be bending of a bending joint or operation of an end effector.

In accordance with a further aspect of the invention, a robotically controlled medical device includes an elongate shaft having a proximal end and a distal end, an end effector at the distal end of the elongate shaft, at least one bending joint along the elongate shaft, between the proximal end and the distal end, a force transmission system at a proximal end of the elongate shaft, having a pushing actuator adapted and configured to be pushed distally by a robotic instrument controller, a pivot, and a reverse linkage rotatable about the pivot, the reverse linkage having a first portion extending between the pivot and the pushing actuator, and operably engaged with the pushing actuator, a second portion extending from the pivot away from the first portion, adapted to engage a control wire of the robotically controlled medical device and pull the control wire proximally in response to distal movement of the pushing actuator, and a distal surface of the second portion having an arcuate surface adapted to maintain an axial position of the control wire throughout a range of motion thereof.

The robotically controlled medical device can further include at least two reverse motion devices associated with each of at least two respective control wires, adapted and configured to antagonistically operate a first motion and a second opposing motion of a function of the robotically controlled medical device. The function can be bending of the at least one bending joint or operation of the end effector.

In accordance with a further aspect of the invention, a control system for a robotically controlled medical device includes a physician console having at least one hand control device, a system controller, a patient cart having at least one instrument controller adapted and configured to operably engage the robotically controlled medical device, the robotically controlled medical device having an elongate shaft having a proximal end and a distal end, an end effector at the distal end of the elongate shaft, at least one bending joint along the elongate shaft, between the proximal end and the distal end, a force transmission system at a proximal end of the elongate shaft, having a pushing actuator adapted and configured to be pushed distally by a robotic instrument controller, a pivot, and a reverse linkage rotatable about the pivot, the reverse linkage having a first portion extending between the pivot and the pushing actuator, and operably engaged with the pushing actuator, a second portion extending from the pivot away from the first portion, adapted to engage a control wire of the robotically controlled medical device and pull the control wire proximally in response to distal movement of the pushing actuator, and a distal surface of the second portion having an arcuate surface adapted to maintain an axial position of the control wire throughout a range of motion thereof.

In accordance with still a further aspect of the invention, a method of controlling a robotic system includes receiving a control input signal from a hand controller, processing the control input signal by a system controller to produce an output control signal, and outputting the output control signal to a robotic instrument controller having a pair of linear actuators arranged in an antagonistic push-push configuration, wherein each linear actuator of the pair of linear actuators is adapted and configured to push a respective reverse motion mechanism, each reverse motion mechanism adapted to convert push actuation into pull actuation of a respective control wire of a robotic instrument, wherein the force transmission linkage includes an arcuate surface adapted to maintain an axial position of the control wire throughout a range of motion thereof.

The processing step can include a scaling calculation. The processing step can include correlation of input control signal and output control signal. The input control signal can be based on a position of a hand control device relative to its mechanical range. An output control signal can be based on mechanical range of a function of the robotic instrument. The method can further include calculating a movement of a respective control wire based on an actuation distance of the linear actuator according to the formula: ΔL=R₂*tan⁻¹(ΔZ/R₁), wherein ΔL is a movement of the control wire, R₂ is a distance between a pivot of the force reverse linkage and the control wire, ΔZ a change in the position of the linear actuator, and R₁ is a linear distance between the pivot and a translation axis of the linear actuator.

The method can further include calculating a tensile force applied to a control wire based on a pushing force applied by the linear actuator, according to the formula: T=(F*R₁)/R₂, wherein T is the tensile force applied to a control wire, F is the pushing force applied by the linear actuator, R₁ is a linear distance between the pivot and a translation axis of the linear actuator, and R₂ is a distance between a pivot of the reverse linkage and the control wire.

In accordance with still a further aspect of the invention, a computer-readable medium for a robotic surgical system is provided, the computer-readable medium storing instructions that, when executed by a computer, cause the computer to: receive a control input signal from a hand controller, process the control input signal to produce an output control signal, and output the output control signal to a robotic instrument controller having a pair of linear actuators arranged in an antagonistic a push-push configuration, wherein each linear actuator of the pair of linear actuators is adapted and configured to engage a respective reverse motion mechanism, adapted to convert push actuation into pull actuation of a control wire of a robotically controlled medical device, wherein the force transmission mechanism includes an arcuate surface adapted to maintain an axial position of the control wire throughout a range of motion thereof.

These and other features of the embodiments of the subject disclosure will become more readily apparent to those skilled in the art from the following detailed description taken in conjunction with the drawings.

### BRIEF DESCRIPTION OF DRAWINGS

So that those skilled in the art to which the subject disclosure appertains will readily understand how to make and use the devices, systems and methods of the subject disclosure without undue experimentation, embodiments thereof will be described in detail herein below with reference to certain figures, wherein:

FIG. 1A is an elevation view of an embodiment of a force transmission system in accordance with this disclosure;

FIG. 1B is an elevation view of the embodiment of FIG. 1A, shown having the actuator shown in phantom to illustrate the pulley assembly in a retracted position;

FIG. 1C is an elevation view of the embodiment of FIG. 1A, shown having the actuator extended;

FIG. 1D is a plan view of the embodiment of FIG. 1A showing a plurality of pulley assemblies and actuators configured to actuate a plurality of cables;

FIG. 2A illustrate another embodiment of a force transmission system in accordance with this disclosure, shown in a first position of actuation;

FIG. 2B illustrate the embodiment of FIG. 2A, shown in a second position of actuation;

FIG. 2C is a cutaway view of a surgical instrument with end effector, incorporating a force transmission system in accordance with the embodiment of FIG. 2A, illustrating a neutral position of an attached end effector;

FIG. 2D is a cutaway view of the surgical instrument of FIG. 2C, illustrating actuation of one joint in one direction;

FIG. 2E is a detail view of the force transmission system of the instrument of FIG. 2C in a neutral position, also illustrating an axis along which the control wires are maintained throughout their range of motion;

FIG. 2F is a detail view of the force transmission system of the instrument of FIG. 2C clearly illustrating antagonistic actuation of one joint in a first of two opposed directions as in FIG. 2D, and also illustrating an axis along which the control wires are maintained throughout their range of motion;

FIG. 3A is an illustration of a lever mechanism in accordance with the prior art utilizing a straight beam to convert pushing force to pulling force, shown in a neutral position;

FIG. 3B is an illustration of the mechanism of FIG. 3A, illustrating radial deviation of control wires from a neutral position at extreme limits of their range of motion;

FIG. 4A is a side view of the reverse motion device of FIG. 2B overlayed with markings illustrating the geometry of thereof;

FIG. 4B is an isometric view of a force transmission system in accordance with the invention, illustrating a plurality of reverse motion devices and control wires;

FIG. 5A is a side view of the reverse motion device in accordance with the embodiment of FIG. 2A;

FIG. 5B is a radially inner isometric view of the reverse motion device of FIG. 2A;

FIG. 5C is a radially outer isometric view of the reverse motion device of FIG. 2A;

FIG. 5D is a distal isometric view of the reverse motion device of FIG. 2A, illustrating a control wire and connection thereof to the reverse motion device;

FIG. 5E is a radial outer view of the distal aspect of the reverse motion device of FIG. 2A;

FIG. 5F is a radial inner view of the proximal aspect of the reverse motion device of FIG. 2A;

FIG. 5G is an exploded side isometric view of the reverse motion device of FIG. 2A;

FIG. 6 is a schematic view of an embodiment of a robotic surgical system in accordance with the invention including a surgeon console and patient cart;

FIG. 7A is an isometric view of an instrument controller in accordance with the present invention;

FIG. 7B is an isometric view of an instrument controller in accordance with the present invention, illustrating an instrument aligned therewith for connection thereto;

FIG. 8 is a distal isometric view of a distal end portion of a steerable overtube in accordance with the invention, including a plurality of instruments extending through the distal end thereof, as well as a videoscope therefor; and

FIG. 9 is a schematic diagram illustrating control flow between a surgeon console and an instrument in accordance with the systems of the present invention.

### DETAILED DESCRIPTION OF THE INVENTION

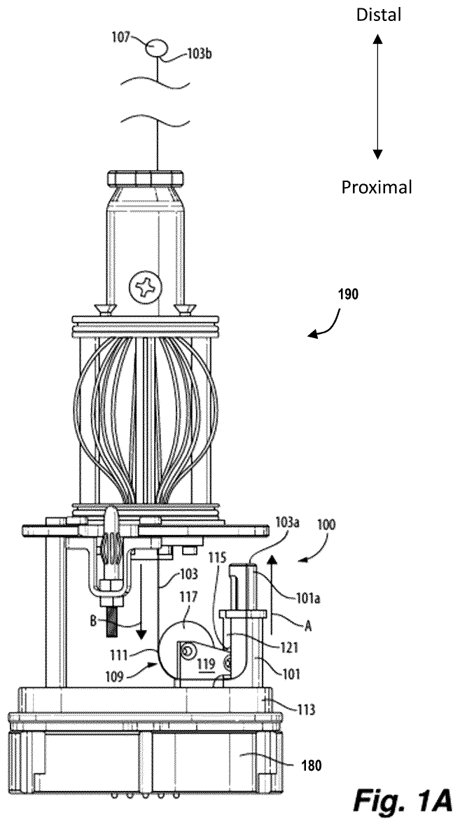

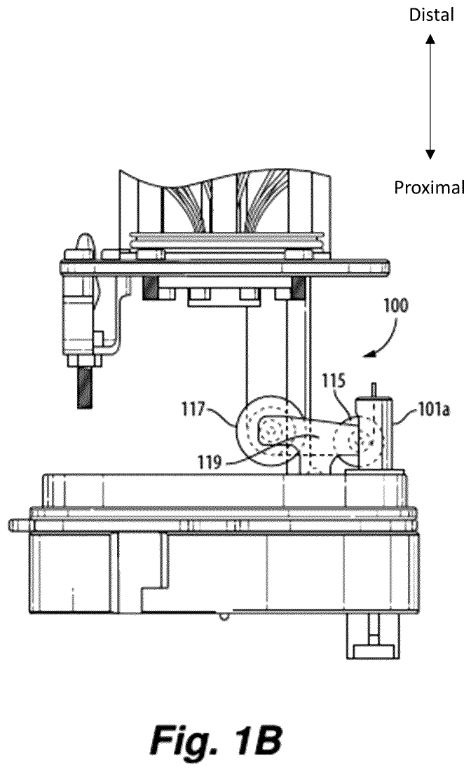

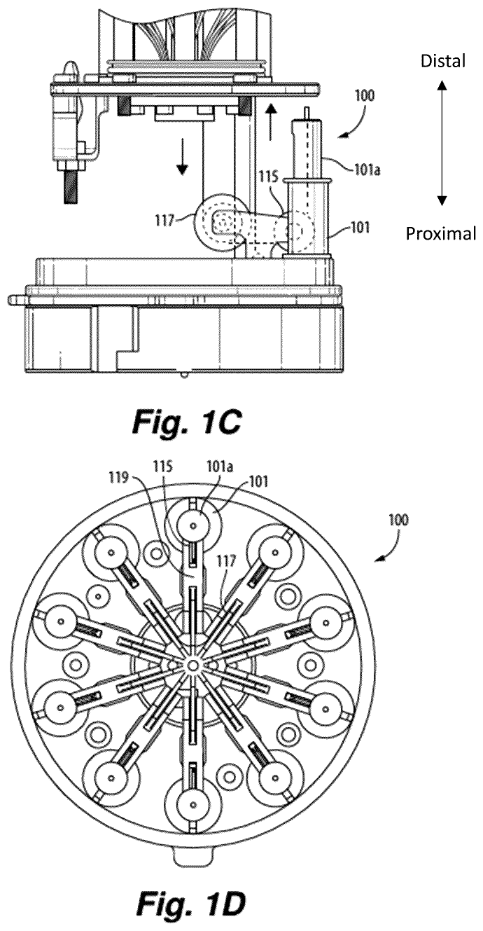

Reference will now be made in detail to the drawings. For the purposes of explanation and illustration, and not limitation, an illustrative view of an embodiment of a force transmission system in accordance with the disclosure is shown in FIG. 1A and is designated generally by reference character 100. Other embodiments and/or aspects of this disclosure are shown in subsequent figures.

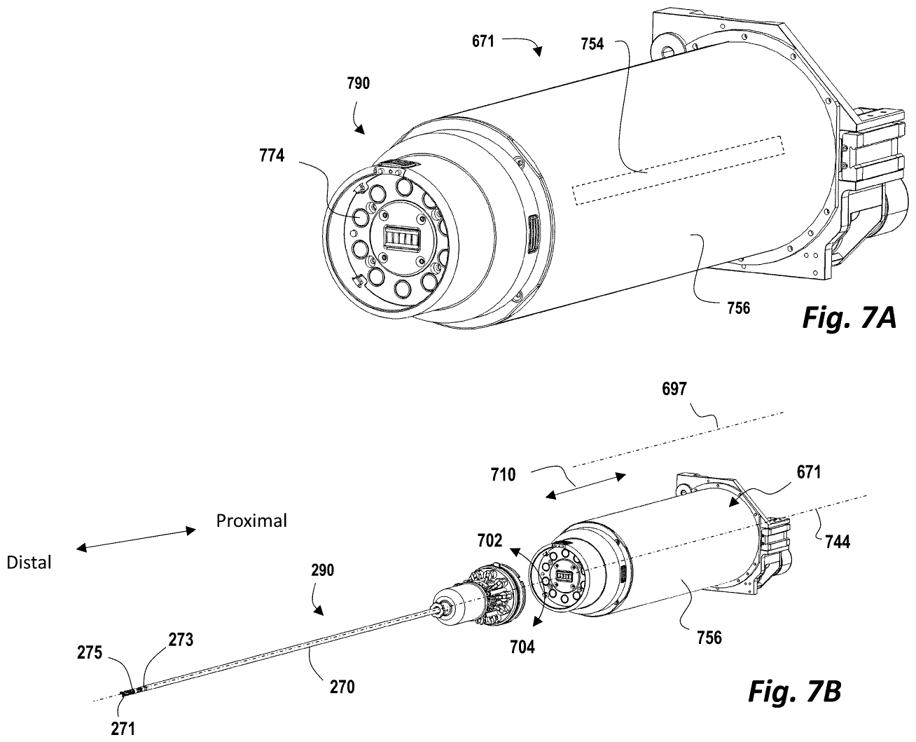

With reference to FIGS. 1A, 1B, 1C, and 1D, in accordance with at least one aspect of this disclosure, a force transmission system 100 for a robotically controlled instrument, such as robotic instrument 190, can include a pushing actuator 101 configured to be pushed by a robotic instrument controller (as will be described in further detail below in connection with FIGS. 7A and 7B) and a control wire 103. The control wire 103 can include a first end 103a attached to an end 101a of the pushing actuator 101 to be pushed distally (e.g., upward as shown in FIG. 1A) by the pushing actuator 101. The control wire 103 can include a second end 103b attached to a distal location 107 of the robotic instrument (e.g., anchored within a steerable assembly to cause steering of a shaft of the robotic instrument, or actuation of an end effector thereof).

The system 100 can include a reverse motion device 109 that can be interfaced with the control wire 103 between the first end 103a and the second end 103b. The reverse motion device 109 can be configured to cause a proximal pulling action on the second end 103b in response to pushing of the first end 103a distally by the pushing actuator 101. The reverse motion device 109 can be configured to maintain a point of contact 111 with the control wire 103 in the same spatial location (e.g., a fixed point relative to the base 113 of an instrument adapter 180 of the robotic instrument) to prevent control wire motion (e.g., radial movement) due to actuation, as will be described in further detail below.

In accordance with this aspect, in certain embodiments the reverse motion device 109 can be one or more pulleys 115, 117 mounted radically and axially fixed to a base 113 (e.g., via a frame 119). The one or more pulleys 115, 117 can be interfaced with the control wire 103 (e.g., such that the pulleys 115, 117 roll when the control wire 103 is actuated). The pushing actuator 101 can be configured to move axially relative to the base 113 (e.g., along direction A shown) to push the control wire 103 (e.g., distally).

In certain embodiments, the one or more pulleys 115, 117 can include a first pulley 115 and a second pulley 117 configured to reverse direction of the control wire 103. In certain embodiments, the first pulley 115 can be inserted within a slot 121 (e.g., defined in the direction of pushing motion, e.g., direction A as shown) of the pushing actuator 101 to cause the control wire 103 to contact the first pulley 115 such that the control wire 103 is parallel and/or coaxial with a pushing axis (e.g., coaxial with the straight portion of the control wire 103. The second pulley 117 can be sized and/or positioned to contact the control wire 103 to be coaxial with a proximal direction axis (e.g., coaxial with the control wire 103 shown in FIGS. 1A-1C, e.g., parallel with direction B). The second pulley 117 can be larger than the first pulley 115, e.g., as shown. In certain embodiments, the reverse direction of the control wire 103 can be 180 degrees, e.g., as shown.

In accordance with at least one aspect of this disclosure, a force transmission system 100 for a robotic instrument can include a pushing actuator 101 configured to be pushed by a robotic instrument controller, and one or more pulleys 115, 117 mounted relative to the pushing actuator 101. The system 100 can include a control wire 103 having a first end 103a attached to an end 101a of the pushing actuator 101 to be pushed distally by the pushing actuator 101, and a second end 103b attached to a distal location 107 of the robotic instrument. The control wire 103 can be interfaced with the one or more pulleys 115, 117 to cause a proximal pulling action on the second end 103b in response to pushing of the first end 103a distally.

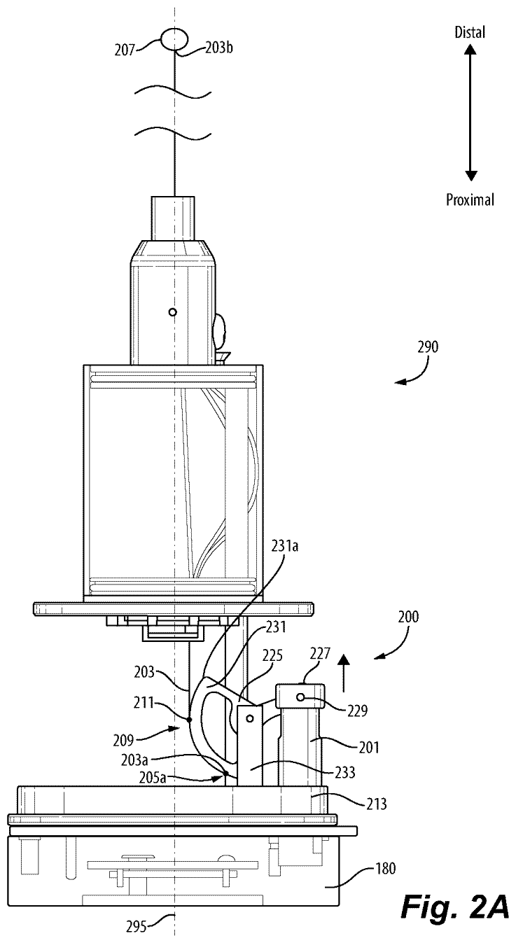

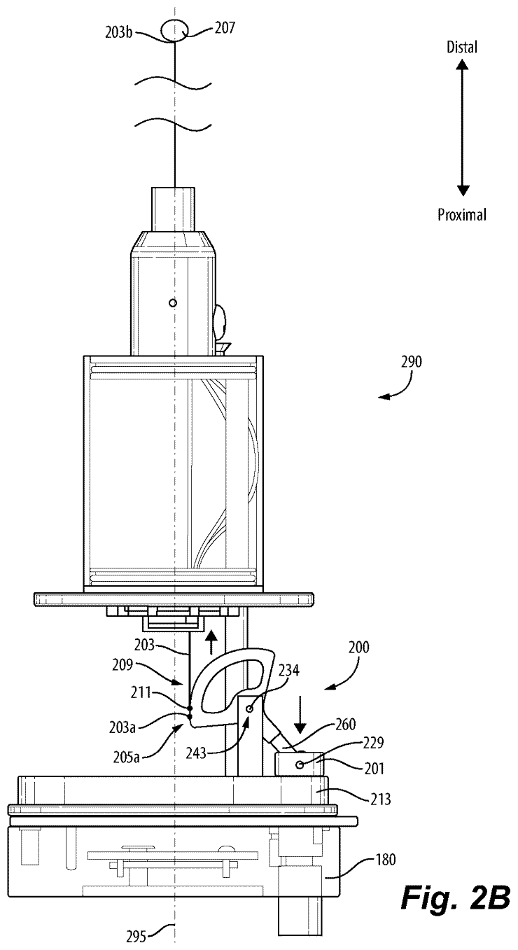

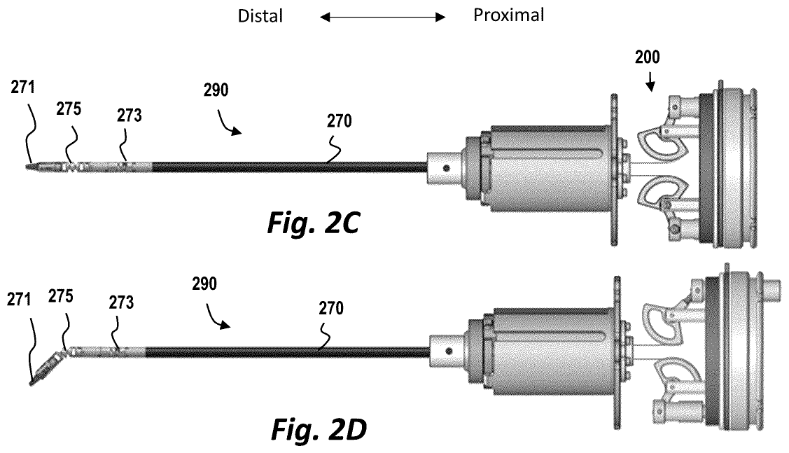

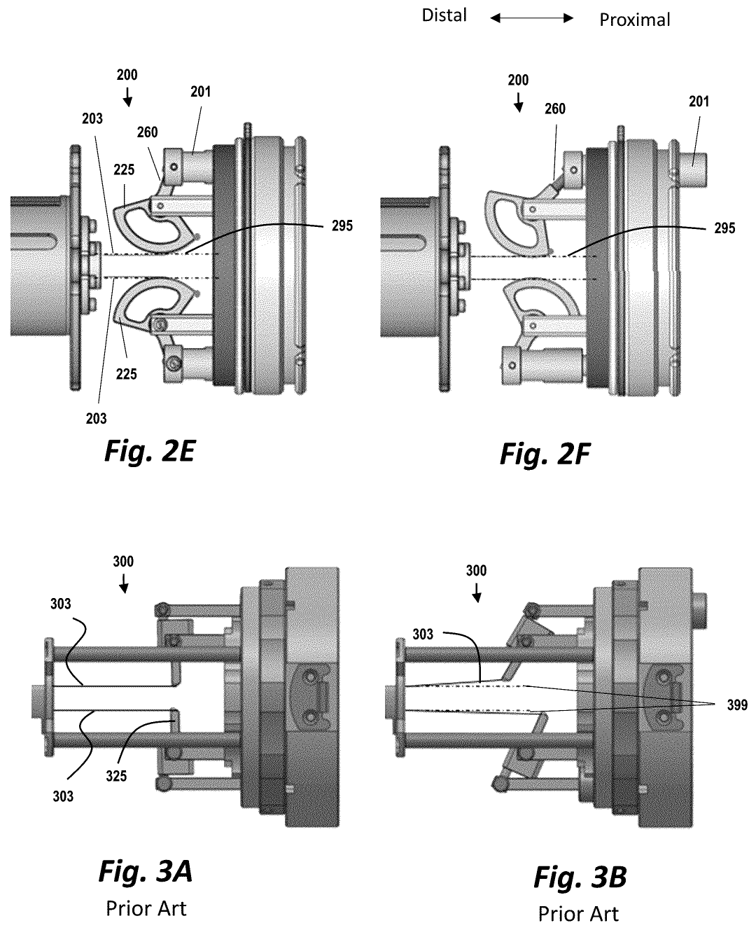

In accordance with at least one further aspect of this disclosure, referring to FIGS. 2A-2F, 4A-4B, and 5A-5G, a force transmission system 200 for a robotic instrument 290, which can be a surgical instrument or medical device, can include a pushing actuator 201 configured to be pushed by a linear actuator of a robotic instrument controller (as will be described in further detail below in connection with FIGS. 7A and 7B) and a control wire 203. The control wire 203 can include a first end 203a attached to a location 205a that moves with movement of the pushing actuator 201. The control wire 203 can include a second end 203b attached to a distal location 207 of the robotic instrument 290 (e.g., anchored within a steerable assembly to cause steering of a shaft of the robotic instrument, or actuation of an end effector thereof).

The force transmission system 200 can include a reverse motion device or mechanism 209 that can be interfaced with the control wire 203 between the first end 203a and the second end 203b. The reverse motion device or mechanism 209 can be configured to cause a proximal pulling action on the second end 203b of the control wire 203 in response to distal pushing by the actuator 201 on the first end 227 of the reverse linkage 225. The reverse motion device 209 can be configured to maintain a point of contact 211 with the control wire 203 in the same spatial location (e.g., a fixed point relative to the base 213 of the instrument adapter 180 of the robotic instrument) to prevent wire motion (e.g., radial movement) due to actuation.

With continued reference to FIGS. 2A-2F, 4A-4B, and 5A-5G, in certain embodiments, the reverse motion device 209 can be a reverse linkage 225 attached to the pushing actuator 201 on a first end 227 (e.g., at a first pin 229) and interfaced with a control wire 203 on the second end 231. The reverse linkage 225 can be rotatably mounted and axially fixed to a base 213 (e.g., at a pivot 243, such as by second pin 234, via frame 233). The second end 231 of the reverse linkage 225 can be or include a curved contact surface 231a to maintain the point of contact 211 with the control wire 203 in the same spatial location (i.e., radial position, with respect to a central axis of the robotic instrument) to prevent control wire motion due to actuation. Any other suitable reverse actuation mechanism is contemplated herein.

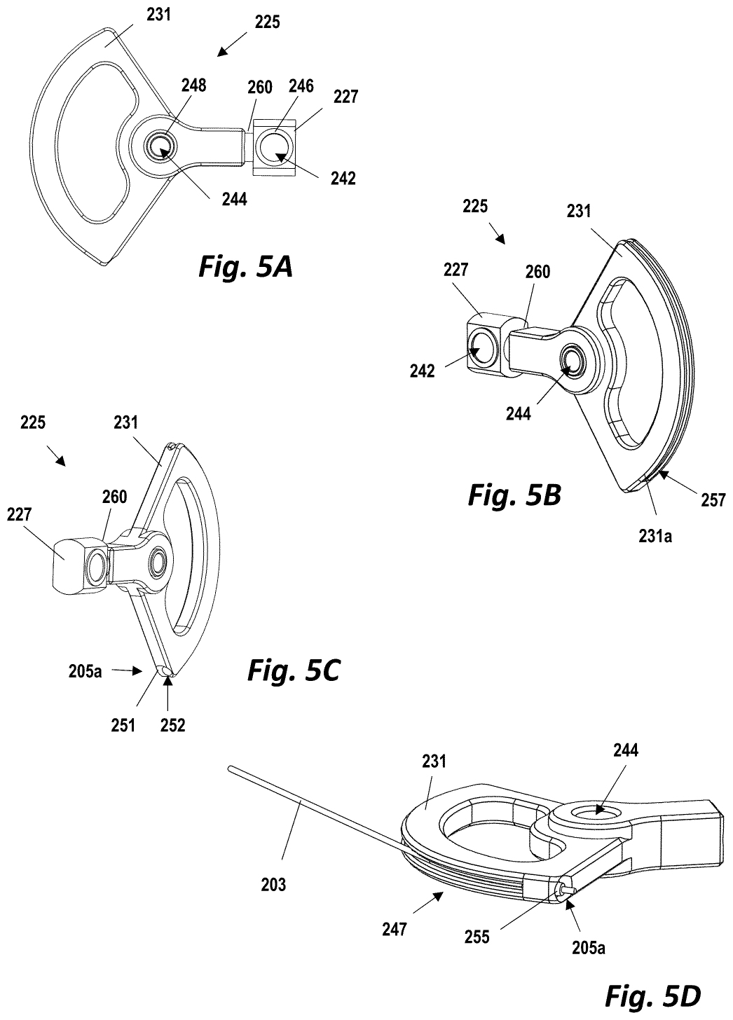

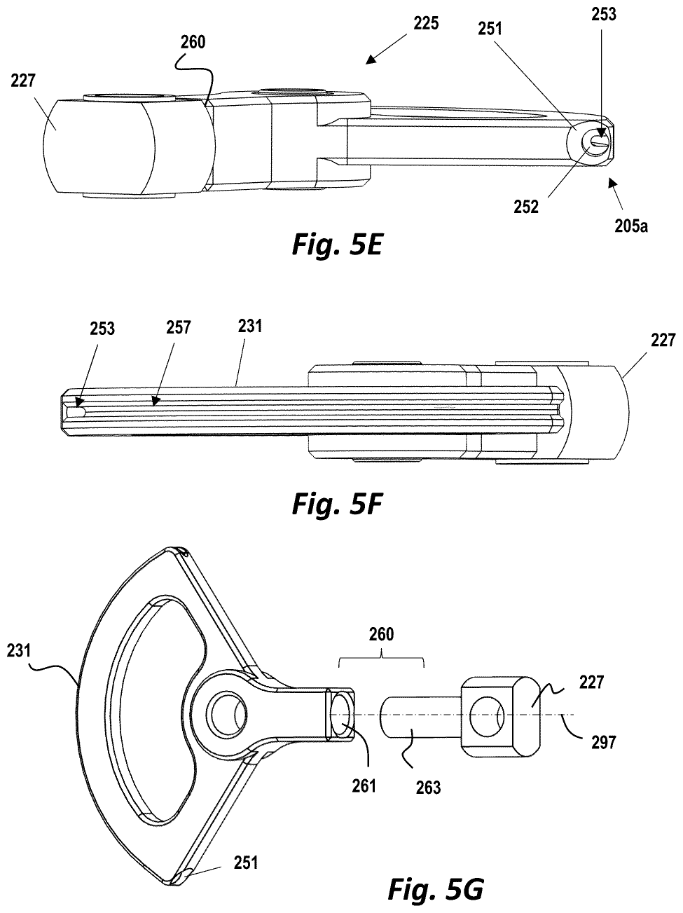

With particular reference to FIG. 2B and FIG. 5G, in the illustrated embodiment, the pushing actuator 201 moves linearly (e.g., parallel to the central axis 295 of the instrument 290), while the portion of the reverse linkage 225 between the pivot 234 and the pin 229 rotates. Accordingly, devices of the present invention can be equipped with a sliding joint 260 to compensate for a changing radius between the pivot 243 and the pin 229 at the pushing actuator 201.

Embodiments in accordance with the invention can include instrument force transmission mechanisms for robotically controlled medical instruments. Embodiments can include a reverse motion design using two fixed pulleys instead of linkages, or a linkage that has a curved surface, for engaging one or more control wires of the robotic instrument. Robotic instruments in accordance with the invention can include any suitable number of force transmission mechanisms (e.g., one for each control wire), jointly comprising a force transmission system.

A robotically controlled instrument can include an adapter having one or more embodiments of a force transmission mechanism in accordance with this disclosure. Any suitable robotic instrument or robotically controlled surgical or medical device (e.g., robotically controlled jaws or blades) is contemplated herein.

In accordance with at least one aspect of this disclosure, a robotically controlled medical device can include a steerable elongate member and a hub (e.g., connected to the steerable elongate member). The robotic instrument can include a force transmission system disposed in the hub. The force transmission system can be or include any suitable force transmission system disclosed herein.

With reference to FIGS. 2C-2F, 4A-4B, and 5A-5G, additional details of the reverse linkage 225 are illustrated alone and in connection with other components of the subject devices, systems and methods.

With reference to FIGS. 2C-2D, cutaway views of a robotic instrument 290, incorporating a force transmission system 200 are illustrated and demonstrate movement of the reverse linkage 225 and control wires 203 in relation to articulation of a second (bending) joint 275 at the distal end of an elongate shaft 270 thereof. Also illustrated are a first bending joint 273 and an end effector 271. Although bending in one plane is illustrated in connection with one pair of antagonistic actuators, their respective reverse motion mechanisms and control wires, it is to be understood that bending at each joint, in each plane, and actuation of an end effector 271 (e.g., graspers, needle driver, scissors) also utilize respective pairs of antagonistic actuators, their respective reverse motion mechanisms and control wires. For example, the second bending joint 275 can be bent downward as illustrated, and also upward, by actuating the opposed control wire, or into or out of the page (not illustrated here), or a combination of those motions, which configuration uses four independent control wires and corresponding reverse motion mechanisms 209 (including reverse linkages 225 and pushing actuators 201) of the force transmission system 200. The first bending joint 273 is preferably provided with the capability of bending in two orthogonal planes, or a combination thereof (i.e. in 3-dimensions), which configuration uses an additional four independent control wires and reverse motion mechanisms 209 of the force transmission system 200. Likewise, opening and closing actuation of an end effector utilizes two additional, respective independent control wires and reverse motion mechanisms 209 operating antagonistically in order to control precise positioning.

With reference to FIGS. 2E-2F, detail views of the transmission system 200 and force reversing linkages 225 and pushing actuators 201 are shown, which correspond, respectively to the positions illustrated in FIGS. 2A-2B. As illustrated, the force reversing linkages 225 of the present invention maintain the radial position of the control wires 203 throughout their range of motion (with respect to a central axis of the instrument 290). Accordingly, displacements can be calculated much more accurately, as compared with use of a straight arm connected to a control wire.

For comparison, prior art embodiments of a force transmission system 300 are illustrated in FIGS. 3A and 3B. FIG. 3A illustrates control wires 303 and their respective levers 325 in a neutral position, while FIG. 3B illustrates each of the two illustrated levers 325 at opposite extents of their range of motion. The deviation of the control wires 303 from a neutral, axially-aligned position 399 is notable and results in challenges to precisely calculating wire movement and applied force and therefore challenges to controlling the functions to which the control wires 303 are connected (e.g., bending joints, end effector).

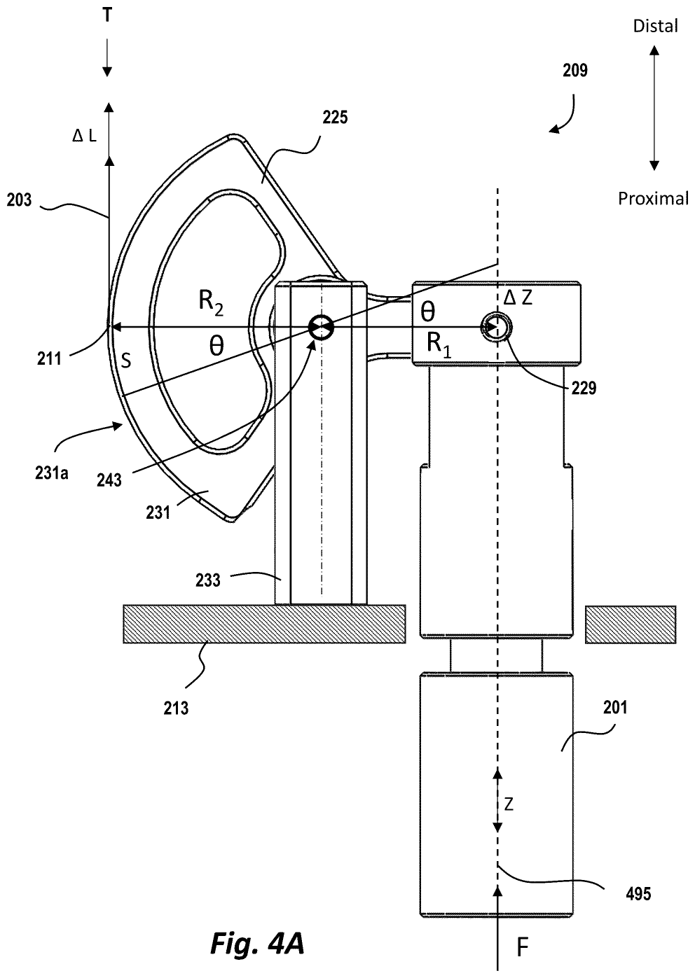

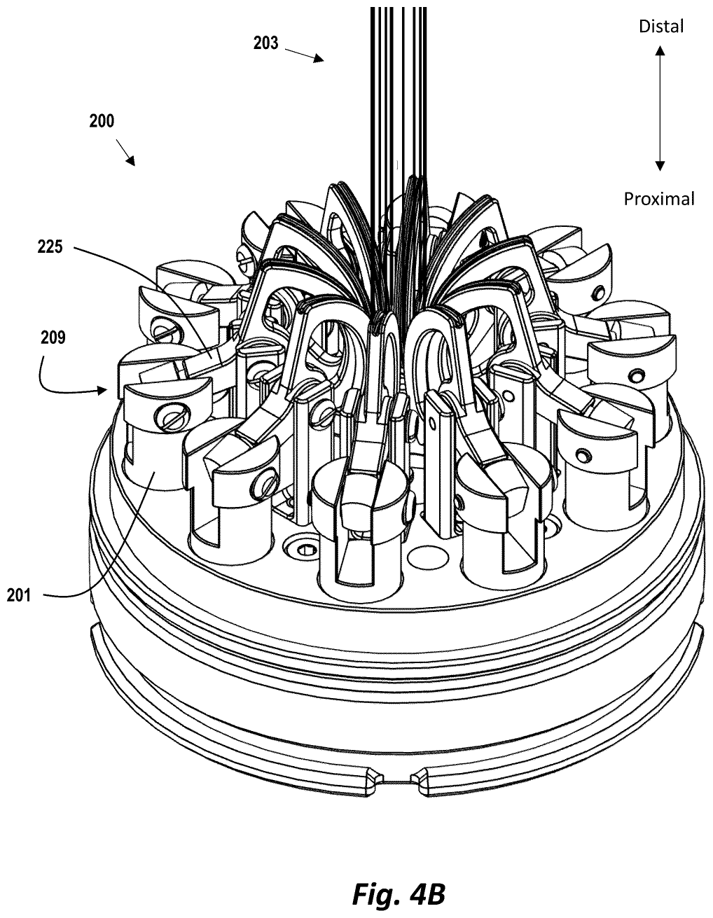

Movement of the control wire 203 and tension T applied thereto are useful parameters for accurately controlling articulations of the subject instruments and actuation of end effectors thereof. In order to illustrate the foregoing benefits of the invention, FIG. 4A shows a reverse motion mechanism 209 of a force transmission system 200 in accordance with the invention, including a pushing actuator 201 and a reverse linkage 225. Also as illustrated, a base 213 and a frame 233 to secure the reverse linkage 225 to the base 213 are provided.

The linear path of the pushing actuator 201 along Z-axis 495 necessitates a linkage with radius (R₁) that varies along its rotational path. However, a change of position along the Z-axis 495 of the pivot 243 is known based on the travel of a corresponding linear actuator of a connected instrument controller (described in more detail below). Accordingly, a rotation angle (θ) of the reverse linkage 225 can be calculated (θ)=tan⁻¹(ΔZ/R₁)) if desired, where the value of R₁ is the linear distance between the pivot and a translation axis of the linear actuator. Similarly, known values, including constant radius R₂, can be used to solve for arc length(S), which is equal to the movement ΔL of the control wire 203 from a distal connection point (e.g. 207 in FIG. 2A) to a the point of contact 211 with the arcuate contact surface 231a of the second end 231 of the reverse linkage 225. Accordingly, the movement ΔL of a respective control wire 203 can be calculated by the subject systems as ΔL=R₂*tan⁻¹(ΔZ/R₁).

Similarly, because radius R₁ at the point in its travel at which it is parallel to the base 213 is a known constant and equal to the distance between the translation axis 495 of the pushing actuator 201 and the pivot 243, and the radius R₂ is also known, a tensile force applied to a control wire based on a pushing force applied by the linear actuator can be calculated by the subject systems as T=(F*R₁)/R₂, where T is the tensile force applied to a control wire, F is the pushing force applied by a linear actuator (described below) and transferred to the pushing actuator 201.

Moreover, a calculated change in position of a distal connection can be further refined utilizing the calculated tension T in conjunction with the material properties (theoretical or measured) of the control wire 203. That is, especially under high tensile force, a proportion of the measured movement ΔL may be attributable to strain (elongation) of the control wire 203 under load.

As will be appreciated in the description below of the detailed views of the reverse linkage 225, the radius (R₂) is calculated between the pivot 243 and the position on the reverse linkage 225 at which the control wire 203 sits. As discussed below in accordance with illustrated embodiments can be in a groove formed on the curved contact surface 231a.

With reference to FIG. 4B, an isometric view of a force transmission system 200 in accordance with the invention is illustrated, having a plurality of reverse motion mechanisms 209 and corresponding control cables 203. In the illustrated embodiment, ten reverse motion mechanisms 209 are illustrated, although it is to be understood that including more or fewer reverse motion mechanisms 209 is well within the scope of the present invention.

With reference to FIG. 5A-5F, detailed views of the reverse linkage 225 of the force transmission system 200 are illustrated, of which FIG. 5D is illustrated in connection with a control wire 203, and FIG. 5G is an exploded view illustrating the sliding joint 260 of the reverse linkage 225.

As illustrated, the reverse linkage 225 includes a groove 257 formed in the face of its curved contact surface 231a to receive and guide the control wire 203. The control wire 203 is secured at an attachment location 205a through an aperture 253 formed in a distal portion of the reverse linkage 225, which aperture 253 is continuous with the groove 257. A ferrule 255 is provided on the control wire 203 to fix the cable 203 to the reverse linkage 225, and can be crimped to the control wire 203 in order to secure it. The ferrule 255 is accommodated in a seat 252 (See FIG. 5E), while further clearance is provided by a recessed area 251. Although a ferrule is illustrated, the devices and systems of the present invention can utilize alternative connection techniques.

The reverse linkage 225 can be provided with a first bore 242 to optionally accommodate a first bushing 246, and adapted to receive the pin 234 joining the reverse linkage 225 to the pushing actuator 201. A second bore 244, optionally accommodating a second bushing 248, is adapted to function as the pivot 243 in conjunction with the frame 233 and a connecting pin, for example.

The first end 227 of the reverse linkage 225 is connected to the second end 231 by a sliding joint 260 to allow the linear motion of the pushing actuator 201 to interface with the radial motion of the reverse linkage 225, as the radius between the pivot 243 and pushing actuator 201 varies. As best seen in FIG. 5G, the sliding joint 260 includes a post 263 attached to the first end 227 portion, and a bore 261 formed in the proximal end 231 portion, adapted to receive the post 263 and to permit relative translation along an axis 297 thereof.

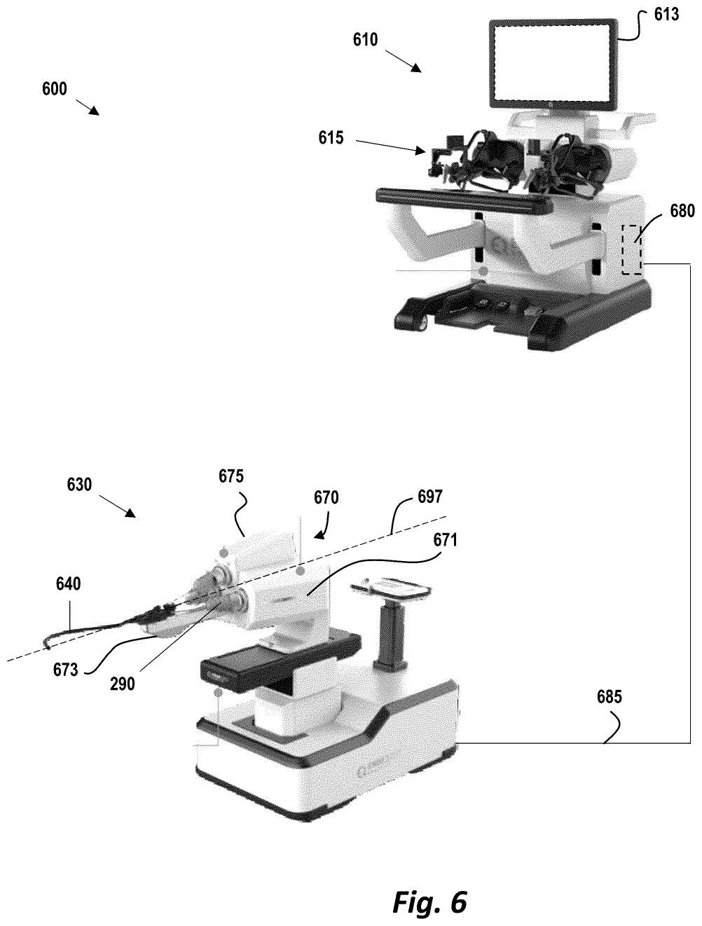

FIG. 6 is an example embodiment of a surgical robotic system 600 in accordance with the invention including an operator or surgeon console 610 operably connected by a system cable 685 to a patient cart 630. Other optional ancillary equipment can be used in accordance with the systems of invention but is omitted herein for simplicity.

The surgeon console 610 includes two hand control devices 615, and a controller 680, which can be a system controller. Although the controller 680 is illustrated in the surgeon console 610, it can be placed in the robotic cart or patient cart 630, housed in a separate device or there can be multiple controllers jointly functioning to control the functions of the subject systems. The surgeon console 610 also includes a display 613 for monitoring and controlling functions of the system 600, displaying images from a videoscope of an operative or working site, and the like. Control outputs 920 can be transmitted from the controller 680 through system cable 685 to the robotic cart 630

The patient cart 630 allows mobility and adjustment of the working components of the subject system 600, and includes one or more instrument controllers 671 for engaging and driving the instruments 190, 290 described above and a videoscope controller 675 for driving a videoscope. The instruments 190, 290, as well as videoscope, are all accepted in working channels of a steerable overtube 640. The overtube controller 673 provides driving force for manipulation of the steerable overtube 640, in addition to manual controls. As will be described in further detail below, the instrument controllers 671 provide driving force for individual control wires 203 of the subject instruments 190, 290 to actuate corresponding functions, such as bending joints (e.g., 273, 275) and operating end effectors (e.g., 271).

In accordance with one aspect of the invention, the instrument controllers 671, videoscope controller 675 and overtube controller 673 are configured to move as one central drive unit 670 to facilitate gross positioning of the steerable overtube 640, along with attached instruments and accessories. An axis 697 of the central drive unit 670 is illustrated for reference along which the central drive unit 670 can translate, and about which it can rotate.

Moreover, the instrument controllers 671 and videoscope controller 675, as will be discussed in further detail below, are adapted to move axially and rotationally with respect to the central drive unit 670 and steerable overtube 640, in particular by axial translation and rotation.

In use, the pushing actuators 101, 201 of instruments 190, 290 are imparted driving force and motion by respective linear actuators of a corresponding instrument controller to which they are attached, such as the instrument controller 671 of FIGS. 7A and 7B.

FIG. 7A is an isometric view of one example of an instrument controller 671 having a plurality of linear actuators 754 in accordance with the present invention. For simplicity, only one linear actuator 754 is illustrated in broken line. FIG. 7B is an isometric view of the instrument controller 671 also illustrating an instrument 290 aligned therewith. A drape adapter 790 is also illustrated on the distal end of the instrument controller 671. The drape adapter interfaces between the instrument controller 671 and the instrument 290, allowing for mechanical passthrough of linear driving force of each of the linear actuators while maintaining a sterile barrier by virtue of being removable and sterilizable, and also by being adapted and configured to secure a sterile drape to the patient cart 630.

As illustrated in FIG. 7A in accordance with one exemplary embodiment, the instrument controller 671 includes multiple linear actuators 754 in such a number as to correspondingly actuate each pushing actuator 201 of the reverse motion mechanisms 209 of the subject force transmission systems 200. Such linear actuators can include lead screw mechanisms or other linear motion devices.

In accordance with one example, in order to move individual reverse motion mechanisms 209, and the corresponding control wires 203 coupled to bending joints 273, 275 or end effector 271, individual linear actuators dedicated respectively thereto are arranged in connection with a supportive structure and/or housing 756. Each linear actuator can include a lead screw housing physically connected to and grounded against rotation and axial movement by the supportive structure or housing, a motor, a threaded shaft configured with outer threads thereon, and a connecting bracket having a first connector portion and/or other ancillary components. The first connector portion can have internal threads into which the threaded shaft extends. An extension member can be provided and can extend distally therefrom in a direction away from a corresponding motor. A second connector portion can then be provided at the end of the extension member, distal to the respective motor. Thus, linear pushing force is output from each linear actuator to an instrument. In the illustration of FIG. 7A, this force is transmitted through the drape adapter 790 by way of individual pushing couplings 774.

In some embodiments, the connecting bracket is movable in the axial direction of the threaded shaft in response to rotational motion of the threaded shaft, but rotation is inhibited with respect to the housing through a pawl received in and axially slidable with respect to a respective groove provided in surface of the housing or supportive structure.

As illustrated, ten linear actuators and corresponding push couplings 774 are provided on the drape adapter 790, actuated by corresponding elements of the instrument controller 671, which are evenly circumferentially spaced from one another within the housing 756. The second connector portion extends from the lead screw housing and into a bore of the instrument controller 671. Each second connector portion is adapted to ultimately impart pushing force on a respective pushing actuator 201 of a corresponding reverse motion mechanism 209, and is controlled to do so in response to system commands generated by the system controller 680. An intervening push coupling 774 of a drape adapter 790 can be provided to transmit the force of each actuator while maintaining a sterile barrier.

Referring now to FIG. 7B, in accordance with one embodiment, the instrument controller 671 and thus the surgical instrument 290 connected thereto are moveable in the axial direction 710 by movement of the housing 756 in the axial direction 710, which can be held in a further housing, of the central drive unit 670, for example. The instrument controller 671 is also rotatable about centerline 744, such as by a rotating a ring gear connected thereto and driven by rotation of a drive motor. This allows controllable rotation of the surgical instrument 290, in either of opposed rotational directions 702, 704. The larger assembly of the central drive unit 670 may also be controllably rotated about axis 697, such that housing 756 held therein will move about axis 697, along with any attached instrument 290.

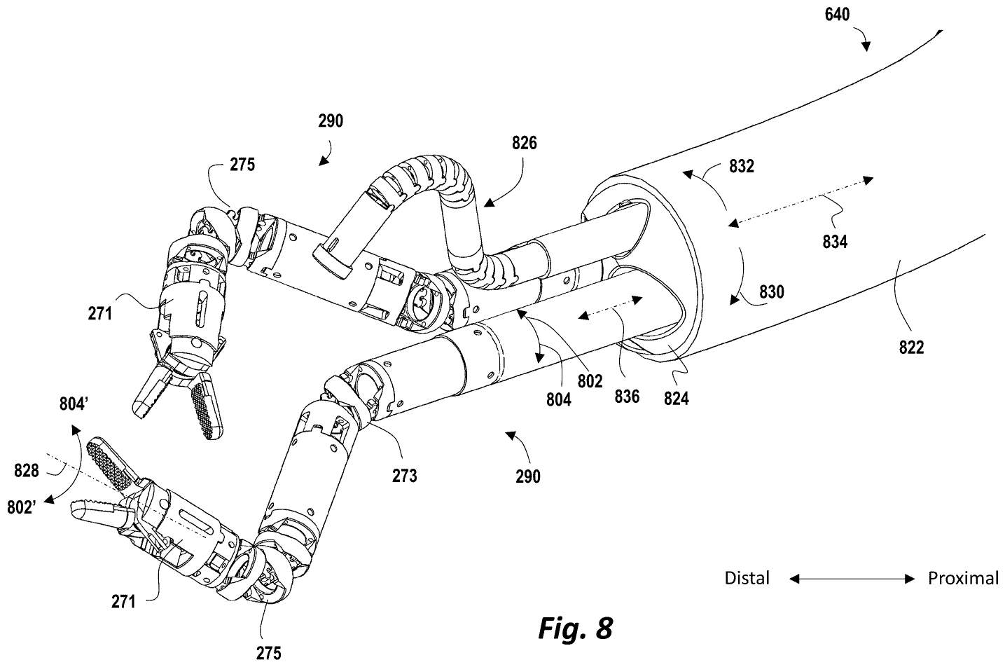

With reference to FIG. 8, a distal isometric view of a distal end portion of a steerable overtube 640 in accordance with the invention is illustrated, which includes a plurality of instruments 290 extending through the distal end thereof, as well as a videoscope 826 therefor. The distal end of a steerable overtube 640 is shown, wherein an outer sheath 822 encloses two instruments 290 and a videoscope 826, each of which is extendable from a cap 824 having openings defined therein through which the instruments 290 and videoscope 826 are selectively extendable. As illustrated, the distal ends of the instruments 290 extend outwardly of the cap 824, and thus the first joint 273 and second joint 275 of each instrument 290 are positioned outwardly of the outer sheath 822, and the end of the videoscope 826 is likewise extended outwardly of the cap 824 and thus of the sheath 822.

If the instrument controller 671 (FIG. 7A, 7B) is rotated in the directions 702, 704 about their centerline 744 (FIG. 7B), the distal ends of the instruments 290 likewise rotate at the cap 824 in directions 802, 804. If the end effector 271 is positioned as shown in FIG. 7B, it will likewise rotate about its own centerline 836 (FIG. 8) in directions 802, 804. However, as the first joint 273 and second joint 275 of each instrument 290 are bendable by selective pulling of control wires 203, the centerline 828 of the end effector 271 and the centerline of the distal end of the instruments 290 can be controllably offset from each other by a single angle when one of the first joint 273 and second joint 275 bend, or a compound double angle when both the first joint 273 and second joint 275 bend.

Additionally, the sheath 822 and thus the instruments 290 and the videoscope 826 therein can be advanced or retracted along direction 834 and rotated about axis 710 (FIG. 7B) and thus rotate in directions 830, 832. Additionally, each instrument 290, and thus the end effector 271 attached thereto, is independently moveable in axial direction 836 by independent movement of the housing to which it is coupled, in translation directions 710. Accordingly, an operator (i.e., technician or surgeon) can position the distal end (the cap 836 end) of the sheath 822 in a desired working area or operative space (e.g., a location in a body lumen), and then with the videoscope 826 view the operative space, including the end effectors 271. Because each of the first joint 273 and second joint 275 are each independently bendable in two orthogonal planes, and the overtube 640 is rotatable about axis 834, the end effector can be positioned in a multitude of orientations in the operative space.

That is, each of the instruments 290, benefit from many degrees of freedom, including translation (e.g., along axis 836), rotation (about axis 836 in opposing directions 802, 804) and 4-way-bending at each of two joints 273, 275. In combination with gross positioning afforded by the steerable overtube 640, this allows the operator to reach difficult locations to successfully complete delicate and complex tasks. The large degree of freedom afforded to the end effectors 271 by way of the first joint 273 and second joint 275 allow triangulation of the end effectors 271 in order to perform tasks in a natural fashion while monitoring progress through the videoscope 826, the image of which is transmitted through the system 600 (e.g. by system cable 685) to the display 613.

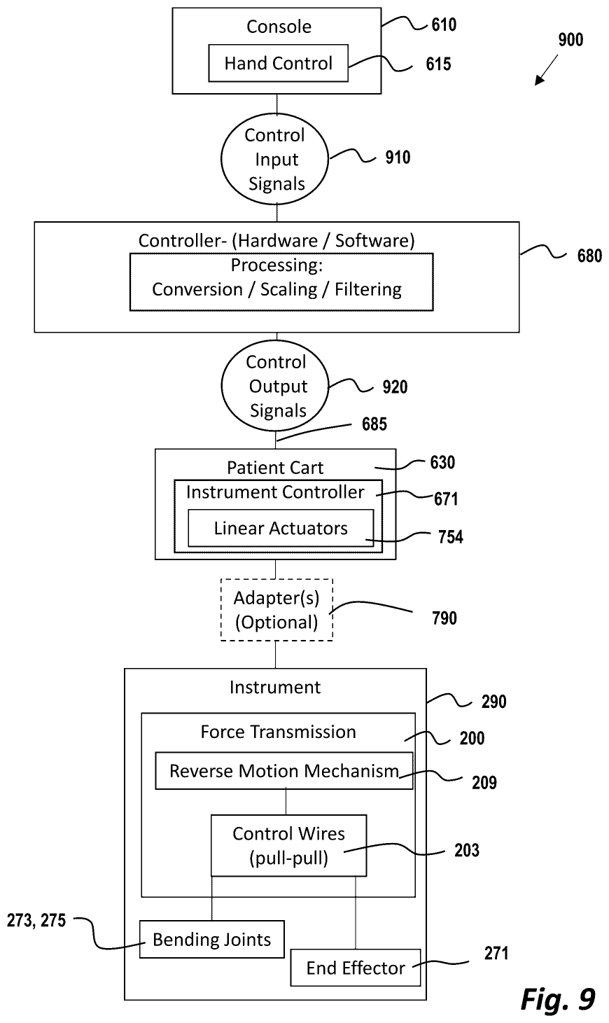

With reference to FIG. 9, each of the four bending directions at each of the joints 273, 275, along with each of an open and close motion of the end effectors 271 is designated a control wire 203, which is actuated through applied tension via the above-described reverse motion mechanisms 209 as part of a force transmission system 200. In turn, each reverse motion mechanisms 209 is actuated by a designated linear actuator of an instrument controller 671. Optionally, one or more adapters, such as sterile drape adapters 790 or other interface can be applied between the instrument controller 671 and the instrument 290.

The linear actuators are selectively controlled by control output signals 920 from a controller 680 in order to effect the desired motion of the instrument 290. The controller 680 generates the control output signals 920 in response to control input signals 910 from the console 610. The control input signals 910 are processed by the controller 680 to interpret command inputs, including change of position of a corresponding hand control device 615 of the console 610. Processing of the control signal can include a scaling function, which can be adjustable by the operator or surgeon. If desired the processing can also include filtering or smoothing of a control input signal 910 to remove unintentional or undesired motion, such as small involuntary movements (e.g. shaking), or sudden and/or large movements, which may indicate an error (e.g., accidental bumping of a controller).

The controller 680 may be embodied as hardware and/or software, but regardless may be referred to herein as a controller, control module and/or system controller. A single physical or software controller can be provided to control all aspects or the system, or alternatively multiple physical or software controllers, such as master controllers and slave controllers can be provided, for example.

When connecting an instrument 290 to the robotic instrument controller 671, an instrument identification process can be initiated by the controller 680 upon connection of the instrument 290 with the instrument controller 671. Alternatively, instrument identification can be performed manually by an operator or other user. In accordance with one example, information assigned to the instrument 290, is conveyed to the controller 680.

Such information can include an identifier, instrument type, calibration data, prior use data or the like. Alternatively or additionally, the information can simply be an identifier or unique identifier for which the controller 680 searches a local or remote database to retrieve relevant data. Subsequently the controller 680 can map an individual linear actuator to a corresponding reverse motion mechanism 209, and in that way, by control wire 203 to the specific function thereof (bend joint e.g., 273, 275 and bend direction (e.g., up, down, left, right) or end effector 271 movement (e.g., open/close)). The controller 680, also mapping the input control signals from a hand control device 615 to a function (e.g., bend up, or close end effector) then matches the input command to the appropriate linear actuator to achieve the desired function.

Gross positioning of the steerable overtube 640 is accomplished by movements of the patient cart 630 itself and adjustable elements thereof for supporting and positioning the central drive unit 670, while smaller adjustments of bending of the distal end portion of the steerable overtube 640 is accomplished through actuation of the overtube controller 673, which interfaces with the bending mechanism of the steerable overtube 640. The subject instruments 290 are adapted to extend through working channels of the steerable overtube 640 and therefore gross positioning (including translation, rotation and bending) of the distal end portions of the surgical instruments 290 is achieved by positioning the distal end of the steerable overtube 640.

In all cases, processing of control signals by the controller 680 can include scaling of control input to actuator output, which scaling can be preprogrammed, and/or input or adjusted by an operator.

In all cases of the described antagonistic control mechanisms, even though one actuator may be activated to perform a bending or actuation function, an opposed actuator can be driven to a position in order to maintain force applied to the opposing reverse motion device 209 and cable 203 in order to stabilize a desired degree of bending (of a joint) or actuation (of an end effector).

With certain illustrated embodiments of the systems, devices and methods described above, it is to be appreciated that various non-limiting embodiments described herein may be used separately, combined, or selectively combined for specific applications. Further, some of the various features of the above non-limiting embodiments may be used without the corresponding use of other described features. The foregoing description should therefore be considered as merely illustrative of the principles, teachings, and exemplary embodiments of this invention, and not in limitation thereof.

Any controller, control module, or other module(s) disclosed herein can include any suitable hardware and/or software module(s) configured to perform any suitable function(s) (e.g., as disclosed herein, e.g., as described above). As will be appreciated by those skilled in the art, aspects of the present disclosure may be embodied as a system, method or computer program product. Accordingly, aspects of this disclosure may take the form of an entirely hardware embodiment, an entirely software embodiment (including firmware, resident software, micro-code, etc.), or an embodiment combining software and hardware aspects, all possibilities of which can be referred to herein as a “circuit,” “module,” or “controller.” A “circuit,” “module,” or “controller” can include one or more portions of one or more separate physical hardware and/or software components that can together perform the disclosed function of the “circuit,” “module,” or “controller”, or a “circuit,” “module,” or “controller” can be a single self-contained unit (e.g., of hardware and/or software). Furthermore, aspects of this disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of this disclosure may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of this disclosure may be described above with reference to flowchart illustrations and/or block diagrams of methods, apparatus (systems) and computer program products according to embodiments of this disclosure. It will be understood that each block of any flowchart illustrations and/or block diagrams, and combinations of blocks in any flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in any flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified herein.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within 1% or within any other suitable percentage or number as appreciated by those having ordinary skill in the art (e.g., for known tolerance limits or error ranges).

The use of the term “substantially” in the Specification and Claims means largely but not wholly that which is specified. The term “substantially” can also mean “consisting essentially of”.

With regard to degree, the term “substantially” in one aspect means greater than 50%, up to and including 100%. The term “substantially” in another aspect means 90% to 100%, inclusive. The term “substantially” in another aspect means 95% to 100%, inclusive. The term “substantially” in another aspect means 97% to 100%, inclusive. The term “substantially” in another aspect means 98% to 100%, inclusive. The term “substantially” in another aspect means 99% to 100%, inclusive. The term “substantially” in another aspect means 99.5% to 100%, inclusive. The term “substantially” in another aspect means 99.6% to 100%, inclusive. The term “substantially” in another aspect means 99.7% to 100%, inclusive. The term “substantially” in another aspect means 99.8% to 100%, inclusive. The term “substantially” in another aspect means 99.9% to 100%, inclusive.

With regard to function and corresponding functional language, the term “substantially” in the Specification and the Claims means sufficiently to such a degree of being precise such that performance of the prescribed action or task, from the perspective of one with ordinary skill in the art, is the same as though the object, element or step were exactly precise.

The term “predetermined” as used herein, including in the Specification and Claims, means an element, quantity or value, for example, that is selected in advance, where precise details, quantities or values can vary, but which nevertheless is relevant to the claimed invention.

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the systems, devices/apparatus and methods of the subject disclosure have been shown and described, those skilled in the art will readily appreciate that changes and/or modifications may be made thereto without departing from the scope of the subject disclosure. For example, those skilled in the art will readily appreciate that the various aspects of the invention described and illustrated throughout the specification, and components thereof, can be readily interchanged with one another and utilized alone or in any combination, without limitation, which is explicitly contemplated herein.

It is to be appreciated that the concepts, systems, circuits and techniques sought to be protected herein are not limited to use in the example applications described herein (e.g., industrial applications, medical/surgical applications), but rather may be useful in substantially any application where the subject devices, systems and methods find advantageous application. While particular embodiments and applications of the present disclosure have been illustrated and described, it is to be understood that embodiments of the disclosure are not limited to the precise construction and compositions disclosed herein and that various modifications, changes, and variations can be apparent from the foregoing descriptions without departing from the spirit and scope of the disclosure as defined in the appended claims.

Accordingly. it is submitted that that scope of this patent should not be limited to the described embodiments but rather should be limited only by the spirit and scope of the following claims.

1. A force transmission system for a robotically controlled medical device comprising:
a pushing actuator adapted and configured to be pushed distally by a robotic instrument controller;
a pivot; and
a reverse linkage rotatable about the pivot, the reverse linkage having:
a first portion extending between the pivot and the pushing actuator, and operably engaged with the pushing actuator;
a second portion extending from the pivot away from the first portion, adapted to engage a control wire of the robotically controlled medical device and pull the control wire proximally in response to distal movement of the pushing actuator; and
a distal surface of the second portion having an arcuate surface adapted to maintain an axial position of the control wire throughout a range of motion thereof.

2. The force transmission system of claim 1, wherein the arcuate surface is adapted to maintain a radial position of the control wire with respect to a central axis of the robotically controlled medical device.

3. The force transmission system of claim 1, wherein the arcuate surface is defined by a constant radius from the pivot.

4. The force transmission system of claim 1, wherein the arcuate surface is provided with a groove to guide the control wire and maintain operable engagement therewith.

5. The force transmission system of claim 1, further comprising the control wire, the control wire having first and second ends, a first end thereof being secured to the second portion of the reverse linkage.

6. The force transmission system of claim 5, wherein the control wire is engaged with an aperture formed in the reverse linkage.

7. The force transmission system of claim 1, wherein the pushing actuator is a linear pushing actuator and wherein the first portion of the reverse linkage is provided with a sliding joint between the pivot and a point of engagement with the pushing actuator, adapted to adjust to a changing radius between the pushing actuator and the pivot through a range of motion of the pushing actuator.

8. The force transmission system of claim 1, further comprising a base, the pushing actuator extending through the base and the pivot secured by the base.

9. The force transmission system of claim 1, comprising at least two reverse motion devices associated with each of at least two respective control wires, adapted and configured to antagonistically operate a first motion and a second opposing motion of a function of the robotically controlled medical device.

10. The force transmission system of claim 9, wherein the function is bending of a bending joint or operation of an end effector.

11. A robotically controlled medical device comprising:
an elongate shaft having a proximal end and a distal end;
an end effector at the distal end of the elongate shaft;
at least one bending joint along the elongate shaft, between the proximal end and the distal end;
a force transmission system at a proximal end of the elongate shaft, comprising:
a pushing actuator adapted and configured to be pushed distally by a robotic instrument controller;
a pivot; and
a reverse linkage rotatable about the pivot, the reverse linkage having:
a first portion extending between the pivot and the pushing actuator, and operably engaged with the pushing actuator;
a second portion extending from the pivot away from the first portion, adapted to engage a control wire of the robotically controlled medical device and pull the control wire proximally in response to distal movement of the pushing actuator; and
a distal surface of the second portion having an arcuate surface adapted to maintain an axial position of the control wire throughout a range of motion thereof.

12. A control system for a robotically controlled medical device, comprising:
a physician console having at least one hand control device;
a system controller;
a patient cart having at least one instrument controller adapted and configured to operably engage the robotically controlled medical device, the robotically controlled medical device comprising:
an elongate shaft having a proximal end and a distal end;
an end effector at the distal end of the elongate shaft;
at least one bending joint along the elongate shaft, between the proximal end and the distal end;
a force transmission system at a proximal end of the elongate shaft, comprising:
a pushing actuator adapted and configured to be pushed distally by a robotic instrument controller;
a pivot; and
a reverse linkage rotatable about the pivot, the reverse linkage having:
a first portion extending between the pivot and the pushing actuator, and operably engaged with the pushing actuator;
a second portion extending from the pivot away from the first portion, adapted to engage a control wire of the robotically controlled medical device and pull the control wire proximally in response to distal movement of the pushing actuator; and
a distal surface of the second portion having an arcuate surface adapted to maintain an axial position of the control wire throughout a range of motion thereof.

13. A method of controlling a robotic surgical system, the method comprising:
receiving a control input signal from a hand controller;
processing the control input signal by a system controller to produce an output control signal; and
outputting the output control signal to a robotic instrument controller having a pair of linear actuators arranged in an antagonistic push-push configuration,
wherein each linear actuator of the pair of linear actuators is adapted and configured to push a respective reverse motion mechanism, each reverse motion mechanism adapted to convert push actuation into pull actuation of a respective control wire of a robotically controlled medical device, wherein the force transmission linkage includes an arcuate surface adapted to maintain an axial position of the control wire throughout a range of motion thereof.

14. The method of claim 13, wherein the processing step includes a scaling calculation.

15. The method of claim 13, wherein the processing step includes correlation of input control signal and output control signal.

16. The method of claim 15, wherein the input control signal is based on a position of hand control device relative to its mechanical range.

17. The method of claim 15, wherein output control signal based on mechanical range of a function of the robotically controlled medical device.

18. The method of claim 13, further comprising:
calculating a movement of a respective control wire based on an actuation distance of the linear actuator according to the formula:
$$Δ L = {R}_{2} * {tan}^{- 1} ( Δ Z / {R}_{1} )$$
wherein ΔL is a change in length of the control wire, R₂ is a distance between a pivot of the force reverse linkage and the control wire, ΔZ a change in the position of the linear actuator, and R₁ is a linear distance between the pivot and a translation axis of the linear actuator.

19. The method of claim 13, further comprising:
calculating a tensile force applied to a control wire based on a pushing force applied by the linear actuator, according to the formula:
$$T = ( F * {R}_{1} ) / {R}_{2}$$
wherein T is the tensile force applied to a control wire, F is the pushing force applied by the linear actuator, R₁ is a linear distance between the pivot and a translation axis of the linear actuator, and R₂ is a distance between a pivot of the reverse linkage and the control wire.

20. A computer-readable medium for a robotic surgical system, the computer-readable medium storing instructions that, when executed by a computer, cause the computer to:
receive a control input signal from a hand controller;
process the control input signal to produce an output control signal; and
output the output control signal to a robotic instrument controller having a pair of linear actuators arranged in an antagonistic a push-push configuration,
wherein each linear actuator of the pair of linear actuators is adapted and configured to correspond to a respective reverse motion mechanism, adapted to convert push actuation into pull actuation of a control wire of a robotic surgical instrument, wherein the force transmission mechanism includes an arcuate surface adapted to maintain an axial position of the control wire throughout a range of motion thereof.
